SEQUENCE LISTING (14 sequences)

SEQ ID NO: 1 - protein, 273 aa, Staphylococcus aureus
  MISC_FEATURE (1)..(273): diaminopimelate epimerase (SAV2470)
MNRQVIEFSKYNPSGNMTILVHSKHDASEYASIANQLMAATHVCCEQVGFIESTQNDDGNDFHLVMSGNEFCGNATMSYIHHLQESHLLKDQQFKVKVSGCSDLVQCAIHDCQYYEVQMPQAHRVVPTTINMGNHSWKALEIIYETYVHYVIPVKQVTTEIQHLVEAFVREQQWSHKYKTVGMMLFDEQRQFLQPLIYIPEIQSLIWENSCGSGTASIGVFNNYQRNDACKDFTVHQPGGSILVTSKRCHQLGYQTSIKGQVTTVATGKAYIE

SEQ ID NO: 2 - protein, 272 aa, Staphylococcus aureus
  MISC_FEATURE (1)..(272): supposed dehydrogenase (SAV2469)
MNNFNNEIKLILQQYLEKFEAHYERVLQDDQYIEALETLMDDYSEFILNPIYEQQFNAWRDVEEKAQLIKSLQYITAQCVKQVEVIRARRLLDGQASTTGYFDNIEHCIDEEFGQCSITSNDKLLLVGSGAYPMTLIQVAKETGASVIGIDIDPQAVDLGRRIVNVLAPNEDITITDQKVSELKDIKDVTHIIFSSTIPLKYSILEELYDLTNENVVVAMRFGDGIKAIFNYPSQETAEDKWQCVNKHMRPQQIFDIALYKKAAIKVGITDV

SEQ ID NO: 3 - protein, 433 aa, Staphylococcus aureus
  MISC_FEATURE (1)..(433): DUF2338 (SAV2468)
MSKLLMIGTGPVAIQLANICYLKSDYEIDMVGRASTSEKSKRLYQAYKKEKQFEVKIQNEAHQHLEGKFEINRLYKDVKNVKGEYETVVMACTADAYYDTLQQLSLETLQSVKHVILISPTFGSQMIVEQFMSKFSQDIEVISFSTYLGDTRIVDKEAPNHVLTTGVKKKLYMGSTHSNSTMCQRISALAEQLKIQLEVVESPLHAETRNSSLYVHPPLFMNDFSLKAIFEGTDVPVYVYKLFPEGPITMTLIREMRLMWKEMMAILQAFRVPSVNLLQFMVKENYPVRPETLDEGDIEHFEILPDILQEYLLYVRYTAILIDPFSQPDENGHYFDFSAVPFKQVYKNEQDVVQIPRMPSEDYYRTAMIQHIGKMLGIKTPMIDQFLTRYEASCQAYKDMHQDQQLSSQFNTNLFEGDKALVTKFLEINRTLS

SEQ ID NO: 4 - protein, 263 aa, Pseudomonas aeruginosa
  MISC_FEATURE (1)..(263): PA4836
MQGRTPLLETLRELECEIRLLTVYARECCGCYEILRRKLDRLSGLIGEDCSRAQWQADSDDPALQALGLRLRDAAVQALCELEKHLCQGVLHEPGEMGRYLGSLLESIRGELDSAGIDADARVLFVGSGALPTSALVLAREVGAHLCCLDIDEEALGCAREIARCQGLEARMQFSSLPPAELAFSRDATHFLIASLVQQKSAVLAQIRQVMRADAKVLLRHGSGIKGLFNYPVEPAELDGWRVCAERVSQPLYDTLILEKAGR

SEQ ID NO: 5 - protein, 433 aa, Pseudomonas aeruginosa
  MISC_FEATURE (1)..(433): PA4835
MNAADESLGNVLLVGLGAVAIQVALDLRRHGAGRLGALNHPGRRSQRIAEALARGACLQLEGQGQHRWLSGNAALDVFHQDPAELRDDWQTLVLCVPADSYLDVVRGLPWERLGGVRTLLLVSAFIGANLLVRSALPAGCQATVLSLSSYYAATKVIDETQPLRALTKAVKRRVYLGSSRPDCPARETWRRVLAGSGVEVVPLATPEAAEGRNVTTYVHSPFFLGEFALARILSEQGPPGFMYKLYPEGPITPGAIGAMRRLWCELSELLRRMGAEPLNLLRFLNDDNYPVHETMLPRASIDGFAEAGAERQEYLLFVRYAALLVDPFSPADEQGRHFDFSAVPFRRVSRDEDGLWRLPRVPLEDYRKLALIVALAAHFDLAMPQARSLLASYENAVSRFIDCQGASQCHPSLYPIDSRPAADAIYRQWCSTC

SEQ ID NO: 6 - DNA, 36 bp, Artificial Sequence
  primer
actagtctagaagcttagcctgaccctgaactactg

SEQ ID NO: 7 - DNA, 34 bp, Artificial Sequence
  primer
agaactagtggatcctgaaggtgaaggacgccag

SEQ ID NO: 8 - DNA, 36 bp, Artificial Sequence
  primer
actagtctagaagcttaccaactgcataagagcctc

SEQ ID NO: 9 - DNA, 36 bp, Artificial Sequence
  primer
agaactagtggatccgatgcaagtaacattgcactc

SEQ ID NO: 10 - DNA, 1059 bp, Rhodopseudomonas palustris
atgaccgatctcgttctgcccgcttcttccgatggattccgcttccgcctggtcgcggtgctcggcggcctggtgctcgccaatatcgccgcctgggcctgggcgctgaccgcgttcgcgggcgagccggtgctgatcggcaccgcggttctggcctatagccttggcctgcgccacgcgctcgacgccgaccatatcgcggcgatcgacaacgtcacccgcaagctgatgcaggagggcaagcggccggtcgcggtcgggctgttcttcgcgctcggccattccaccgtcgtgctggtggcgtcgctggcgatcgcagtcgcggccaattcgctgaccgaacggttctcgggctatcgcgagatcggcggggtgatcggcacctcggcgtcggcgctgtttctgttcgcgatcgccatcgccaatctgtcggtgctgagcggcgtgtatcgggcgttcaagaaggccaaggccggcgaggtcgtgcacgacgaagacatcaacgcgctgctgcagcagcgcggctggctggcgcggctgttccggccgctgttccgcttcgtctcgaagagctggcagttgtttccgatcgggctattgttcgcgctcggcttcgagaccgccagcgaaatcagcctgttcggactggccgccaacgcctccggcagcatttcgcactggacgatcctggtgttcccggcgctgtttgccgccggtatgaccctggtcgacaccctcgacggcgtgctgatgctcggcgcctatggctgggcgtatcgcaatccgatccgcaagctgtactacaacatgacgatcacgacggtgtcggtgctggtggcgctgctgatcggcggcatcgagacgctcggactgctcgccgggcgcttccatctcgaaggcgcgttctgggagtggatcgaagaactcaattccaatttcggcgcgctcggttacggcatcgtggcgctgttcgtggcgagctggatcgtctccaccatcatctatcgcctcaacggctatcaccggctcgaccaggcagccgcggccgtgcagaaatga

SEQ ID NO: 11 - DNA, 37 bp, Artificial Sequence
  primer
ctcgaggagctcaagcttttccaatgaccaccaccac

SEQ ID NO: 12 - DNA, 30 bp, Artificial Sequence
  primer
gtcgacggatccactagtctgatctccggc

SEQ ID NO: 13 - DNA, 28 bp, Artificial Sequence
  primer
actagtggatccatgaccgatctcgttc

SEQ ID NO: 14 - DNA, 28 bp, Artificial Sequence
  primer
ggtaccgaattctcatttctgcacggcc

(19) United States
(12) Patent Application Publication
(10) Pub. No.: US 2019/0085338 A1
(43) Pub. Date: Mar. 21, 2019

(54) Modified Magnetotactic Bacteria Expressing a Metallophore Specific for Cobalt and/or Nickel

(71) Applicant: Commissariat a I'Energie Atomique et aux Energies Alternatives, Paris (FR)

(72) Inventors: David Pignol, Manosque (FR); Monique Sabaty, Saint Martin de La Brasque (FR); Pascal Arnoux, Reillanne (FR); Jean-Baptiste Abbe, Pertuis (FR)

(73) Assignee: Commissariat a l'Energie Atomique et aux Energies Alternatives, Paris (FR)

(21) Appl. No.: 15/767,065

(22) PCT Filed: Nov. 7, 2016
(86) PCT No.: PCT/EP2016/076856

§ 371 Date: Apr. 9, 2018

(30) Foreign Application Priority Data
Nov. 6, 2015 (EP) 15306769.9

(51) Int. Cl. C12N 15/52; C12N 15/70; C12N 15/74; C12Q 1/02; A61K 35/74; A61K 49/00

(52) U.S. Cl. CPC C12N 15/52; C12N 15/70; A61K 49/0002; C12Q 1/02; A61K 35/74; C12N 15/74

(57) ABSTRACT

The invention concerns magnetotactic bacteria modified to express metallophores and their use in bioremediation, biodetection, imaging, as well as the use of magnetosomes extracted from such bacteria in several indications including antitumor treatment and in a process of metal recovery.

### SEQUENCE LISTING SUBMISSION VIA EFS-WEB

A computer readable text file, entitled “SequenceListing.txt,” created on or about Jun. 13, 2018 with a file size of about 18 kb contains the sequence listing for this application and is hereby incorporated by reference in its entirety.

The present invention relates to bacteria engineered to synthesize compounds which increase their ability to resist as well as to take up cobalt and/or nickel from their environment. More specifically, the invention concerns magnetotactic bacteria modified to express metallophores and their use in bioremediation, biodetection, imaging, as well as the use of magnetosomes extracted from such bacteria in several indications including antitumor treatment and in a process of metal recovery.

Magnetotactic bacteria (or MTB) are a polyphyletic group of Gram-negative bacteria discovered by Richard P. Blakemore in 1975. They passively align and actively swim along the geomagnetic field and other magnetic fields. This unique feature is based on specific intracellular organelles, the magnetosomes, which, in most MTB, comprise nanometer-sized, membrane bound crystals of magnetic iron and are organized into chains via a dedicated cytoskeleton.

Because of the special properties of the magnetosomes, MTB are of great interest for paleomagnetism, environmental magnetism, biomarkers in rocks, magnetic materials and biomineralization in organisms; bacterial magnetite has been exploited for a variety of applications in modern biological and medical sciences.

MTB can be found in freshwater and salt water, and in oxygen rich as well as anoxic zones at depths ranging from the near-surface to 2000 meters beneath the surface. However, the majority of MTB discovered so far gather at the so-called oxic-anoxic transition zone. They can be spiral-shaped, rods and spheres.

### DETAILED DESCRIPTION OF THE INVENTION

The present invention concerns a genetically modified magnetotactic bacteria (MTB) expressing a cobalt and/or nickel-specific metallophore.

As used herein, a “cobalt and/or nickel-specific metallophore” is a compound able to form a complex with a cobalt or a nickel ion. Such metallophore may be able to bind cobalt or nickel, or both.

The inventors have previously identified two compounds able to chelate cobalt and nickel. These metallophores are synthesized by two bacteria: Staphylococcus aureus and Pseudomonas aeruginosa, and have been respectively named staphylopine and pseudopaline.

In one embodiment, the genetically modified MTB of the invention produces a molecule of formula (I):

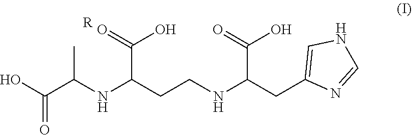

wherein R represents either a methyl group or a propionate group.

Among the molecules of formula (I), two preferred molecules are staphylopine and pseudopaline. Thus, in a preferred embodiment, the invention concerns a genetically modified MTB expressing a metallophore which is staphylopine of formula (II):

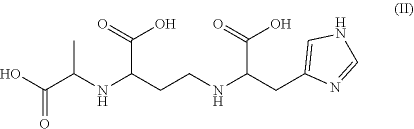 [SMILES: CC(NC(CCNC(Cc1c[nH]cn1)C(=O)O)C(=O)O)C(=O)O]

In another preferred embodiment, the invention concerns a genetically modified MTB expressing a metallophore which is pseudopaline of formula (III):

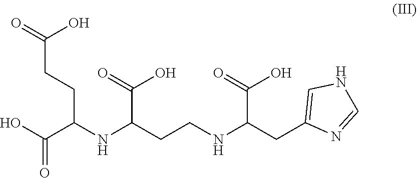 [SMILES: O=C(O)CCC(NC(CCNC(Cc1c[nH]cn1)C(=O)O)C(=O)O)C(=O)O]

The inventors have shown that bacteria able to produce a metallophore can be obtained by introducing the genes responsible for the biosynthesis of said metallophores into the bacteria. In particular, they demonstrated that:
three genes from Staphylococcus aureus are responsible for staphylopine biosynthesis. These genes express the proteins identified in the databases as SAV2470, SAV2469 and SAV2468 and corresponding in the present text to SEQ ID NO: 1, SEQ ID NO: 2 and SEQ ID NO: 3, respectively.
two genes from Pseudomonas aeruginosa are responsible for the pseudopaline biosynthesis. These genes express the proteins identified in the databases as PA4836 and PA4835 and corresponding in the present text to SEQ ID NO: 4 and SEQ ID NO: 5, respectively.

Thus, in a particular embodiment, the invention concerns a genetically modified MTB expressing genes encoding the proteins of Staphylococcus aureus of SEQ ID NO: 1, SEQ ID NO: 2 and SEQ ID NO: 3 or variants thereof. Such a bacterium produces staphylopine. In another particular embodiment, the invention concerns a genetically modified MTB expressing genes coding the proteins of Pseudomonas aeruginosa of SEQ ID NO: 4 and SEQ ID NO: 5 or variants thereof. Such a bacterium produces pseudopaline.

The invention also concerns a genetically modified magnetotactic bacterium characterized in that it expresses a cobalt and/or nickel-specific metallophore, wherein the metallophore is chosen among staphylopine and pseudopaline, and (i) when the metallophore is staphylopine, said bacteria expresses the proteins of SEQ ID NO:1, SEQ ID NO:2 and SEQ ID NO:3 or variants thereof, and (ii) when the metallophore is pseudopaline, said bacteria expresses the proteins of SEQ ID NO: 4 and SEQ ID NO: 5 and variants thereof.

As used herein, the term “variant” corresponds to a sequence which differs by at least one amino acid from the sequence of reference, provided that the function of the protein is retained. An homologous sequence can, for example, be qualified of variant. Also modified or isoform sequences having retained at least one of the properties that make them biologically active are encompassed in the scope of this definition. Typically, a variant sequence presents at least 40%, 50%, 60%, 70%, 75%, 80%, 85%, 90%, 95%, 98% or 99% of identity with the protein of reference, as measured by BLAST method. In a preferred embodiment, a variant sequence presents at least 40% of identity with the sequence of reference. Further, for example, a protein having a sequence identical to SEQ ID NO: 1 and a tag at its N-terminal or C-terminal extremity is a variant of SEQ ID NO: 1, provided that it conserves its activity. For example, a variant of the protein of SEQ ID NO: 1, when co-expressed in a bacterium with the proteins of SEQ ID NOs: 2 and 3, enables the biosynthesis of staphylopine by said bacterium.

It has been previously shown that the two preferred metallophores of the invention, staphylopine and pseudopaline, are able to chelate both cobalt and nickel.

At relatively high concentration, cobalt and nickel are toxics to bacteria. The degree of toxicity is metal-dependent. For example, cobalt is more toxic than nickel.

The inventors of the present invention have demonstrated that expressing these metallophores in magnetotactic bacteria allows to increase their resistance to cobalt and to nickel.

MTB is a large group of bacteria wherein only a limited number have been isolated in pure cultures so far. Among them, Magnetospirillum gryphiswaldense MSR-1, Magnetospirillum magneticum AMB-1, Magnetospirillum magneticum MGT-1, Magnetovibrio MV-1, Magnetococcus sp. MC-1, Marine magnetic spirillum QH-2, Magnetospirillum sp. WM-1 and Magnetospirillum magnetotacticum MS-1 are all affiliated to the α-Proteobacteria; Desulfovibrio magneticus RS-1 is affiliated to the β-Proteobacteria. These and any other MTB can be used in the frame of the present invention.

According to a preferred embodiment, the genetically modified MTB used in this invention are Magnetospirillum gryphiswaldense MSR-1 or Magnetospirillum magneticum AMB-1.

In addition, genetically modified MTB expressing genes responsible for the biosynthesis of a cobalt and/or nickel-specific metallophore from other bacteria than Staphylococcus aureus and Pseudomonas aeruginosa, for example homologous genes from Serratia marcescens or Yesinia pestis, are also part of the invention.

The present invention also concerns MTB which have acquired new properties.

The inventors have demonstrated that genetically modified MTB able to synthesize staphylopine or pseudopaline present unexpected properties relating to their capacity to both resist to metal and accumulate metal.

In particular, a genetically modified MTB of the invention is more resistant to cobalt and/or nickel than the parent magnetotactic strain which does not express the metallophore.

This property is illustrated in the experimental part, especially on FIG. 3A, FIG. 3B, and FIG. 3C, where the MTB expressing a metallophore grow better than the control strain in a medium containing cobalt or nickel. This result was obtained with both M. gryphiswaldense MSR-1 and M. magneticum AMB-1 as starting bacteria, and with both staphylopine and pseudopaline as newly-synthesized metallophore.

A used herein, “a bacteria that is more resistant to metal than the parent strain” corresponds to a bacteria which is able to survive in a medium containing a concentration of metal lethal for the parent strain. Most of the time, this strain is able to grow better than the parent strain when placed in sublethal concentrations of such metal. Such a strain is also a strain which will survive longer than the parent strain in an environment containing metal.

Thus, in a particular embodiment, a genetically modified MTB of the invention which is more resistant to cobalt and or nickel than the parent magnetotactic strain can be a recombinant M. gryphiswaldense MSR-1 or M. magneticum AMB-1.

In another particular embodiment, a genetically modified MTB of the invention which is more resistant to cobalt and/or nickel than the parent magnetotactic strain synthesizes staphylopine or pseudopaline.

In a further embodiment, a genetically modified MTB of the invention which is more resistant to metal than the parent magnetotactic strain is indeed more resistant to cobalt.

In a further embodiment, a genetically modified MTB of the invention which is more resistant to metal than the parent magnetotactic strain is indeed more resistant to nickel.

In a further embodiment, a genetically modified MTB of the invention which is more resistant to metal than the parent magnetotactic strain is indeed more resistant to both cobalt and nickel.

In another aspect of the invention, a genetically modified MTB of the invention accumulates higher quantity of cobalt and/or nickel than the parent strain.

In a particular embodiment, a genetically modified MTB of the invention exhibit a cobalt or nickel accumulation capacity that is at least 20% superior to the cobalt accumulation capacity of the parent strain which does not produce the metallophore.

In a particular embodiment, a genetically modified MTB expresses pseudopaline and the cobalt accumulation is at least twice higher than the cobalt accumulation in the parent strain (FIGS. 4A and 4B). The cobalt accumulation in these bacteria can even be more than three times higher than in the parent strain (FIG. 4B).

In another particular embodiment, a genetically modified MTB expresses staphylopine and the cobalt accumulation is at least three times higher than the cobalt accumulation in the parent strain.

The cobalt accumulation in these bacteria can even be more than three times higher than in parent strain (FIGS. 4A and 4B).

In another embodiment, a genetically modified MTB expresses pseudopaline or staphylopine and the nickel accumulation is higher than the nickel accumulation in the parent strain (FIG. 4C).

This nickel accumulation is at least 30% higher than the nickel accumulation in the parent strain.

In another aspect of the invention, a genetically modified MTB of the invention accumulates cobalt and/or nickel in the magnetosomes.

In a particular embodiment, such bacteria contain at least 50 ng of cobalt per mg dry weight. In a preferred embodiment, such bacteria contain at least 75 ng of cobalt per mg dry weight, as illustrated in FIG. 5, more preferably 80 or 85 ng of cobalt per mg dry weight, and even more preferably more than 90 ng of cobalt per mg dry weight.

In further embodiment, the invention concerns a recombinant MTB expressing a cobalt and/or nickel specific metallophore and a cobalt and/or nickel permease.

As used herein, a “cobalt and/or nickel permease” is a permease located at the cellular membrane which is specific for cobalt and nickel importation.

The inventors have demonstrated that a recombinant MTB expressing both a metallophore and a cobalt and/or nickel permease presents an improved resistance to metal and a higher accumulation capacity than the parent MTB strain and than the MTB expressing only a metallophore.

In a particular embodiment, the cobalt and/or nickel permease is encoded by the NxiA gene. In a particular embodiment, the NxiA gene is from Rhodopseudomonas palutris and corresponds to the sequence SEQ ID NO: 10.

The NxiA permease belongs to a gene family also retrieved in several bacterial strains as for example in H. pylori, N. aromaticivirans, R. rodochrous and R. pulustris . . . In R. palustris (CGA009 strain), this permease is identified in the public database Cyanobase (http://genome.microbedb.jp/CyanoBase) as “RPA0724 gene” and as corresponding to nxiA (in H. pylori), nixA (in S. aureus), HoxN (in R. rhodochrous) and NhIF (in R. Eutropha). All these genes code for cobalt and/or nickel permeases. The use of these permeases for the bioremediation is known from literature; they can be used in the frame of the invention.

According to the above-described features, a recombinant MTB of the invention thus corresponds a bacteria which expresses a metallophore or to a bacteria which expresses both a metallophore and a cobalt and/or nickel permease.

In another aspect, the invention also concerns magnetosomes extracted from the magnetotactic bacteria of the invention. These magnetosomes are made of a proteo-lipidic membrane surrounding a single crystal of magnetite. The biosynthesized magnetite is of higher purity than chemically synthesized ones and has also a narrow size range of 50-100 nm, which participates to its singular properties when compared to chemically synthesized magnetite.

Thus, in a particular embodiment, the invention concerns a nickel- or cobalt-doped magnetosome isolated from a genetically modified MTB of the invention, especially when isolated from bacteria having accumulated cobalt and/or nickel. Such doped-magnetosomes can thus be extracted from bacteria expressing a metallophore or from bacteria expressing both a metallophore and a cobalt and/or nickel permease.

As used herein, a “doped-magnetosome” according to this invention contains at least 20% more cobalt than a magnetosome from a MTB non-expressing a metallophore. In particular, the quantity of cobalt contained in a magnetosome can be measured by comparison to the quantity of iron; the quantity of iron being not modified by the expression of metallophore, it can be used as a reference to evaluate the accumulation of cobalt or nickel. Using this system, the inventors have shown that the MTB producing staphylopine and/or pseudopaline can accumulate in their magnetosomes a relative quantity of cobalt/iron around 1.3% whereas this ratio is of 1% in non-recombinant MTB (Table 4).

Thus, in one embodiment, the invention concerns a cobalt- and/or nickel-doped magnetosome. Such magnetosome can be defined as presenting a ratio cobalt/iron of at least 1.25.

Further, magnetosomes extracted from bacteria expressing both a metallophore and a cobalt and/or nickel permease contain a higher quantity of cobalt and/or nickel than those extracted from bacteria expressing only a metallophore. Such magnetossome may contain at least 25%, and preferably 30%, preferably 40%, and even more preferably 50% more cobalt than a magnetosome from a parent MTB. Further, they present a ratio cobalt/iron of at least 1.5, more preferably of at least 2.

Another aspect of the invention concerns the use of cobalt- and/or nickel-doped magnetosome isolated from a MTB of the invention in antitumor treatment.

Indeed, bacterial magnetosomes can efficiently be used to generate heat in a solution when exposed to an alternative magnetic field. For anti-tumoral application, magnetosomes can be used as such or encapsulated within a vesicule and possibly targeted by any appropriate means including for example antibody, aptamer, recombinant protein, synthetic molecule . . .

The antitumor treatment can be administered directly to the patient for in vivo treatment. The heat treatment is generated by applying a magnetic field which provokes the production of heat by magnetosomes. The frequency of such magnetic field should lie between about 50 kHz and 1000 kHz, preferably between about 100 kHz and 500 kHz, more preferably between about 100 hKz and 200 kHz. The strength of the magnetic field is comprised between about 0.1 mT and 200mT, preferably between 1 mT and 100 mT, more preferably between about 10 mT and 60 mT.

A person skilled in the art would know how to determine the appropriate characteristic of the magnetic field in order to obtain an efficient heat but without toxic side-effects. The thermotherapy can be optimized by adjusting the different parameters including the amount of magnetosomes administered to the patient, the characteristics of the magnetic field, the duration of the application of the magnetic field and the protocol of the treatment regarding the number of repetitions of the treatment (i.e., one application or repeated ones).

This invention also concerns the use of nickel- or cobalt-doped magnetosome isolated from MTB of the invention, in imaging.

An example of imaging application is now described. The membrane surface of the magnetosomes allows the attachment of specific bacteriophages expressing targeting molecules such as antibodies. In addition, it is possible to rely on the magnetic properties conferred by the magnetism to control the bacteria's moving by applying an alternative magnetic field. Thus, one can surround a defined area using the MTB. If a bacteriophage-magnetosome complex meets a cell or molecule of interest, the magnetosome will stick on it through the bacteriophage. Then, the cell or molecule of interest can be detected by using magnetics crystals as contrast agent. Examples of other applications of MTB in imaging are the direct use of their magnetosomes as a contrast agent. Indeed magnetosomes are ultrasensitive magnetic resonance imaging (MRI) T2-contrast agents.

In a further aspect, the invention concerns the use of a bacterium according to the invention in bioremediation of cobalt and/or nickel.

For almost a century, intense human activities such as mining, chemical industries and intensive agriculture led to high accumulation of toxic metals in the environment. These toxic metals are difficult to remove from the environment, since they cannot be easily degraded and are ultimately indestructible. In this context, the inventors of the present application have proposed an efficient bioremediation process. For example, MTB engineered to produce pseudopaline or staphylopine could be grown in liquid media containing nickel and cobalt at subtoxic levels. Because these bacteria accumulate more metal, they can be used to extract these metals from the liquid solution. Furthermore, in another aspect, the present invention concerns a process of recovery of cobalt and/or nickel contained in the MTB.

The aim of this process is to provide a system which allows an easy recovery of metal present in a liquid medium using a magnet. With this aim, the inventors proposed to use MTB expressing a metallophore to recover metal for the environment.

In a preferred embodiment, the metallophore is staphylopine or pseudopaline and the metal trapped in the magnetosome is cobalt and/or nickel.

In a particular embodiment, a process of recovery of cobalt and/or nickel of the invention comprises the following steps: (i) contacting bacteria according to the invention with a medium containing cobalt and/or nickel, and (ii) after an incubation period, creating a magnetic field to recover bacteria containing cobalt and/or nickel.

The process of recovery of cobalt and/or nickel of the invention can be applied to any liquid medium containing such a metal. In a preferred embodiment, this medium containing cobalt and/or nickel from which this metal is recovered is a medium to be depolluted.

The incubation period can be between 3 hours (accumulation was demonstrated at this short period of time) to 120 Hours (cells begin to suffer and die after this period). A preferred incubation duration can be at least between 24 h and 90 h, for example of 48 h, 60 h or 72 h. A preferred incubation duration is 72 Hours.

The medium to be depolluted can be any liquid medium containing cobalt and/or nickel such as cooling water or radioactive waste from nuclear plants (mainly cobalt) or contaminated sludges from battery factories (mainly nickel).

Another aspect of the invention concerns the use of a recombinant MTB according to the invention as a biodetector for cobalt and/or nickel traces.

Indeed, a bacteria expressing a metallophore has the ability to take up cobalt and/or nickel from the environment and to concentrate it intracellularly. The presence of cobalt and/or nickel can then be detecting for example by introducing a reporter gene placed under the control of a promoter sensitive to cobalt and/or nickel. Such promoter can be for example the promotor controlling the expression of the nikABCDE Ni-uptake operon, or the promotor controlling the rcnAB operon which encodes a Ni and Co efflux system (Cayron J. et al., Environ Sci Pollut Res Int. 2015). According to this embodiment, the recombinant MTB strain of the invention can be used to detect very low quantity of cobalt and/or nickel.

A recombinant strain expressing both a metallophore and a reporter construct comprising a promoter sensitive to cobalt and/or nickel, is also part of the invention.

The invention will now be described in further details using the following non-limiting examples.

### LEGENDS OF FIGURES

FIGS. 1A and 1B: Plasmid constructs containing the expression cassette for genes involved in the biosynthesis of staphylopine or pseudopaline. FIG. 1A) Plasmid pBBR1-MCS2 with two promoters and the three genes responsible for the production of staphylopine (saEND); FIG. 1B) plasmid pBBR1-MCS2 with two promoters and the two genes responsible for the production of pseudopaline (paND).

FIGS. 2A and 2B: Growth curves of various bacterial strains in the absence of metal. FIG. 2A) Strains of M. gryphiswaldense MSR-1 grown in the absence of cobalt, strain control (plasmid pBBR1-MCS2 empty) in circle, strain paND (plasmid pBBR1-MCS2-paND) in triangle, strain saEND (plasmid pBBR1-MCS2-saEND) in square. FIG. 2B) Strains of M. magneticum AMB-1 grown in the absence of cobalt, strain control (plasmid pBBR1-MCS2) in circle, strain paND (plasmid pBBR1-MCS2-paND) in triangle, strain saEND (plasmid pBBR1-MCS2-saEND) in square

FIGS. 3A, 3B and 3C: Growth curves of various bacterial strains in the presence of metal (cobalt 100 μM or nickel 1 mM). FIG. 3A) Strains of M. gryphiswaldense MSR-1 grown in presence of cobalt 100 μM, strain control (plasmid pBBR1-MCS2) in circle, strain paND (plasmid pBBR1-MCS2-paND) in triangle, strain saEND (plasmid pBBR1-MCS2-saEND) in square. FIG. 3B) Strains of M. magneticum AMB-1 grown in presence of cobalt 100 μM, strain control (plasmid pBBR1-MCS2) in circle, strain paND (plasmid pBBR1-MCS2-paND) in triangle, strain saEND (plasmid pBBR1-MCS2-saEND) in square. FIG. 3C) Strains of M. gryphiswaldense MSR-1 grown in presence of nickel 1mM, strain control (plasmid pBBR1-MCS2) in circle, strain paND (plasmid pBBR1-MCS2-paND) in triangle, strain saEND (plasmid pBBR1-MCS2-saEND) in square.

FIGS. 4A, 4B, and 4C: Metal accumulation in magnetotactic bacterial strains producing staphylopine or pseudopaline. FIG. 4A) Measurement of cobalt accumulated per mg of dry weight of M. gryphiswaldense MSR-1 strains exposed to 50 μM of cobalt. Strain control (plasmid pBBR1-MCS2) in open bar, paND (plasmid pBBR1-MCS2-paND) in grey bar and saEND (plasmid pBBR1-MCS2-saEND) in black bar. FIG. 4B) Measurement of cobalt accumulated per mg of dry weight of M. magneticum AMB-1 strains exposed to 50 μM of cobalt. Strain control (plasmid pBBR1-MCS2) in open bar, paND (plasmid pBBR1-MCS2-paND) in grey bar and saEND (plasmid pBBR1-MCS2-saEND) in black bar. FIG. 4C) Measurement of nickel accumulated per mg of dry weight of M. gryphiswaldense MSR-1 strains exposed to 500 μM of nickel. Strain control (plasmid pBBR1-MCS2) in open bar, paND (plasmid pBBR1-MCS2-paND) in grey bar and saEND (plasmid pBBR1-MCS2-saEND) in black bar. Error bars correspond to the standard deviation observed for three biological replicates.

FIG. 5: Analysis of cobalt content in the magnetosomal compartment. Measurement of the cobalt/iron ratio accumulated in the magnetosomes.

FIG. 6: Repartition of cobalt between the cytosolic and magnetosomal fractions Experimental XANES spectra measured on magnetosome and cytosol fractions superimposed with the best linear combination fit in the −30/+85 eV region obtained using various spectra measured on Co-Nicotianamine, Vitamine B12, CoFe₂O₄ and Co₃O₄as references (see Table)

FIG. 7: Construction of the plasmid for rpNxiA expression. A) Map of the plasmid pRK415. B) The pRK415-mam plasmid. C) Final plasmid named pRK415-mam-rpNxia.

FIG. 8: Growth curves of various bacterial strains in the presence of metal (cobalt 100 μM or nickel 1 mM). Strains of M. gryphiswaldense MSR-1. Strain control (pBBR1-MCS2 and pRK415) in open circle and strain control+rpNxiA (pBBR1-MCS2 and pRK415-rpNxiA) in open circle and dotted lines, strain paND (pBBR1-MCS2-paND and pRK415) in open triangle and strain paND+rpNxiA (pBBR1-MCS2-paND and pRK415-rpNxiA) in open triangle and dotted lines, strain saEND (pBBR1-MCS2-saEND and pRK415) in open square and saEND+rpNxiA (pBBR1-MCS2-saEND and pRK415-rpNxiA) in open square and dotted lines. A) Strains grown in presence of 100 μM of cobalt. B) Strains grown in presence of 1 mM of nickel.

FIG. 9: Metal accumulation in magnetotactic bacterial strains producing staphylopine or pseudopaline with or without rpNxiA expression. Measurement of cobalt accumulated per mg of dry weight of M. gryphiswaldense MSR-1 strains exposed to 100 μM of cobalt. A) Strain control (plasmid pBBR1-MCS2+plasmid pRK415) in open bar and Strain control +rpNxiA (plasmid pBBR1-MCS2+plasmid pRK415-rpNxiA) in black bar. B) Strain paND (plasmid pBBR1-MCS2-paND+plasmid pRK415) in open bar and strain paND+rpNxiA (plasmid pBBR1-MCS2-paND+plasmid pRK415-rpNxiA) in black bar C) Strain saEND (plasmid pBBR1-MCS2-saEND+plasmid pRK415) in open bar and strain saEND+rpNxiA (plasmid pBBR1-MCS2-saEND +plasmid pRK415-rpNxiA) in black bar.

### EXAMPLES

### Example 1

### Cloning of the Genes Involved in the Biosynthesis of Pseudopaline and Staphylopine

a. Description of the Genes

Two genes from Pseudomonas aeruginosa (PA4836 and PA4835) are responsible for the pseudopaline biosynthesis. Three genes from Staphylococcus aureus (SAV2470, SAV2469 and SAV2468) are responsible for staphylopine biosynthesis. One of these genes encodes a histidine racemase (SAV2470), two others encode Nicotianamine-like synthases (PA4836 and SAV2469) and finally the two remaining enzymes (PA4835 and SAV2468) encode a member of the DUF2338 family experimentally identified as a N-(CA)amino acid dehydrogenases. These enzymes (and their corresponding genes) are the hallmark of a bacterial metallophore biosynthetic machinery. Hereafter, for clarity, the two genes from Pseudomonas aeruginosa are named « paND » (for P. aeruginosa Nas-like and DUF2338 coding genes) and the three genes from Staphylococcus aureus are named « saEND » (for S. aureus Epimerase, Nas-like and DUF2338 coding genes).

b. Description of Gene Constructs

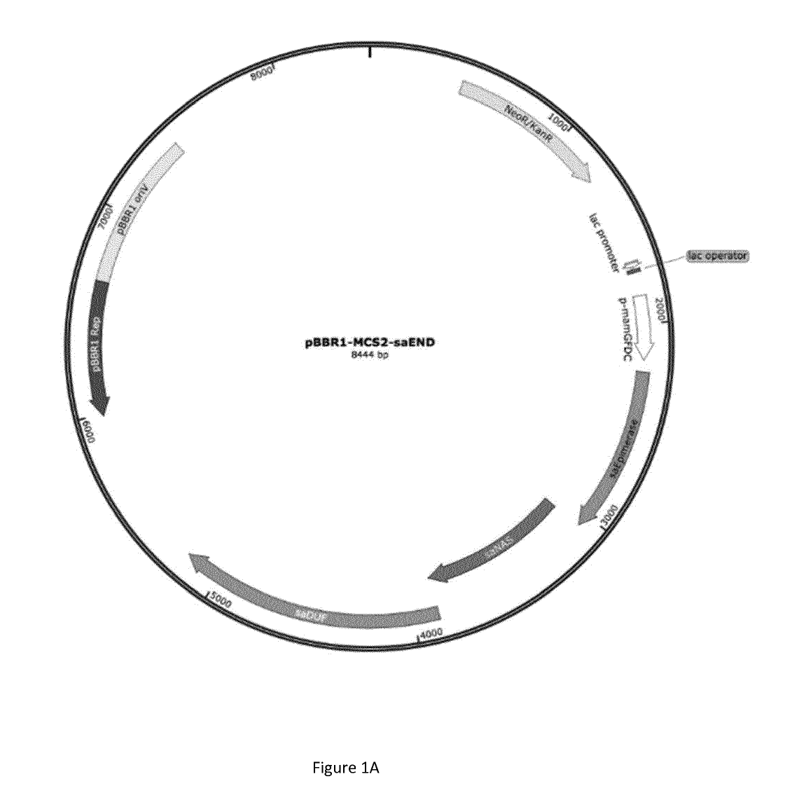

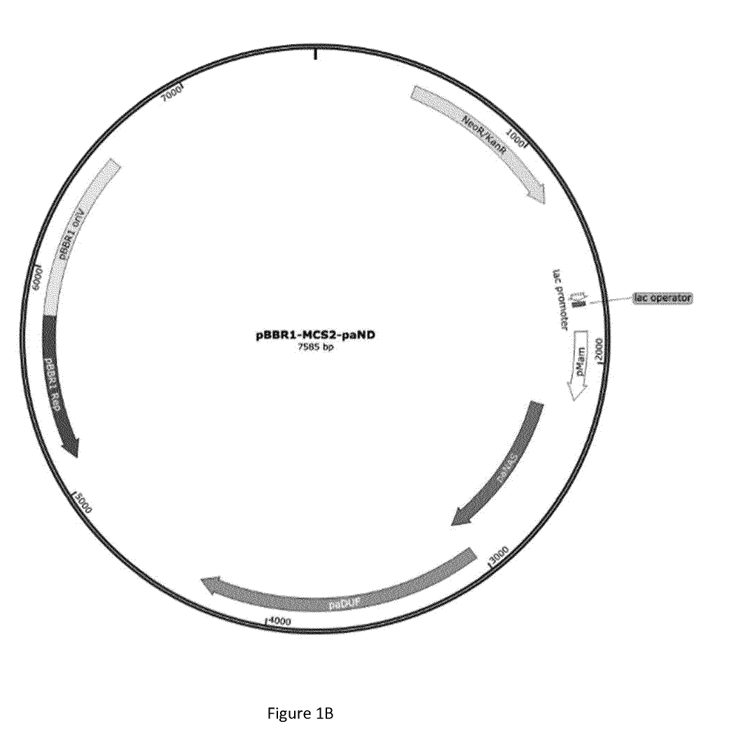

Plasmids have been designed in the laboratory and ordered at Genecust ©. They contain the DNA sequence of the genes from S. aureus Mu50 and P. aeruginosa PA-01 integrated into the broad host plasmid pBBR1-MCS2. The genes have been inserted downstream two promoters: 1/the lac promoter for the expression of the genes in E. coli 2/the promoter mamGFDC of Magnetospirillum gryphyswaldense for the expression of the genes in magnetotactic bacteria. The constructs designed for expressing the genes in magnetotactic bacteria are reproduced in FIG. 1A and FIG. 1B.

### Example 2

### Description of the Organisms, of the Growth Media and Growth Conditions

Transfer of the genetic constructions as described in Example 1, in magnetotactic bacteria (M. gryphyswaldense MSR-1 and Magnetospirillum magneticum AMB-1) has been done by conjugation of the magnetotactic strain using a strain of E. coli previously transformed with the genes constructs and harboring the genes tra required for conjugation. Thus, the strain E. coli WM3064 was chosen for its ability to transfer pBBR1-MCS2 in a large variety of hosts (including magnetotactic bacteria) with a counter-selection in a medium devoid of diaminopimelate, the strain being auxotrophic toward this molecule.

Both 1 mL of overnight culture of magnetotactic bacteria and 200 μL of an overnight culture of E. coli strain WM3064 carrying the pBBR1-MCS2 constructs have been collected and resuspended in 30 μL of appropriate medium (see below) supplemented with diaminopimelate (0.3 mM). The 30 μL of mixed bacteria have been disposed on an agar plate of appropriate medium supplemented with 0.3 mM diaminopimelate, and left at 30° C. for 24 H. The cells have then been collected and plated on solid medium with antibiotic and without diamniopimelate, thus ensuring the selection of magnetotactic bacteria carrying the pBBR1-MCS2 construct, and eliminating the E. coli strain. Magnetotactic strains have then been selected and screened for the plasmid presence and the integrity of the construction by simple PCR amplification of the paND fragment.

Primers used to amplify the paND sequence are the following:

PA-ND-IF-F:
(SEQ ID NO: 6)
ACTAGTCTAGAAGCTTAGCCTGACCCTGAACTACTG
PA-ND-IF-R:
(SED ID NO: 7)
AGAACTAGTGGATCCTGAAGGTGAAGGACGCCAG
SA-END-IF-F:
(SEQ ID NO: 8)
ACTAGTCTAGAAGCTTACCAACTGCATAAGAGCCTC
SA-END-IF-R:
(SEQ ID NO: 9)
AGAACTAGTGGATCCGATGCAAGTAACATTGCACTC

M. gryphyswaldense MSR-1 and M. magneticum AMB-1 have been cultivated respectively in MSR-1 lactate medium pH 7 and MagMin 1.5 medium pH 6.9. MSR-1 lactate: (HEPES 10 mM, Na-lactate 0.15%, Soja-Peptone 0.3% Yeast extract 0.01%, NaNO₃ 4 mM, KH₂PO₄ 0.7 mM, MgSO₄ 0.6 mM, Fe-citrate 50 μM, and 0.1% Trace Element Solution : H₃Bo₃ 162 μM, Na₂MoO₄ 74 μM, ZnSO₄ 250 μM, CuCl₂ 6 μM, NiCl₂ 50 μM, CoCl₂ 400 μM, MnCl₂ 250 μM, Na₂EDTA 7 mM).MagMin 1.5: (KH₂PO₄ 5 mM, NaNO₃ 1.5 mM, Na Acetate 850 μM, Ascorbic acid 0.2 mM, Tarataric acid 2.5 mM, Succinic acid 3.1 mM, Na thiosulfate 0.2 mM, 0.5% Modified Mineral Wolf Elixir:Nitrilotriacetic acid (NTA) 7.8 mM, MgSO₄12.2 mM, MnSO₄ 2.9 mM, NaCI 17 mM, FeSO₄ 360 μM, CoCl₂ 420 μM, CaCl₂ 680 μM, ZnSO₄ 348 μM, CuSO₄ 100 μM, AIK(SO₄)₂ 21 μM, H₃BO₃ 162 μM, Na₂MoO₄ 1.65 mM, NiCl₂41 μM).

All strains have been cultivated under microaerophilic conditions (O₂=2%) at 30° C. with the appropriate antibiotic.

### Example 3

### Growth of the Magnetotactic Bacteria in the Presence of Metal

25 mL of medium supplemented with different metals have been inoculated at a final OD₆₀₀ₙₘ=0.1 for M. gryphiswaldense MSR-1 and OD₆₀₀ₙₘ=0.03 for M. magneticum AMB-1 with an overnight preculture. Growth was then followed by measurement of optical density at 600 nm.

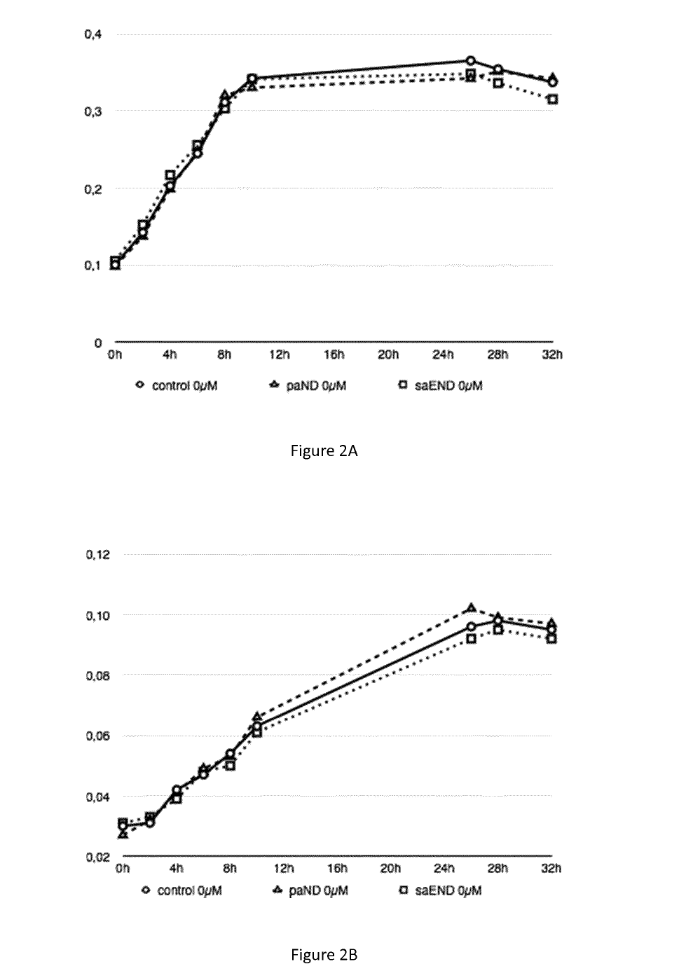

As shown in FIG. 2A and FIG. 2B, the growth of magnetotactic bacteria (MSR-1 in FIG. 2A and AMB-1 in FIG. 2B) is unaffected by the type of plasmid they carry in the absence of metal in the growth media. Thus, the expression of staphylopine or pseudopaline do not modify the growth rate of the magnetotactic bacteria in the absence of metal, which is equivalent to the growth of those bacteria without plasmid.

TABLE 1
Measurement of OD₆₀₀ₙₘ of M. gryphiswaldense MRS-1 parent strain
(Control) or strains expressing pseudopalyne (paND or staphylopine
(saEND) in the presence or absence of cobalt.

| | Control | Control | paND | paND | saEND | saEND |
|---|---|---|---|---|---|---|
| OD₆₀₀ₙₘ | 0 μM | 100 μM | 0 μM | 100 μM | 0 μM | 100 μM |
| 0 | 0.1 | 0.096 | 0.098 | 0.098 | 0.105 | 0.103 |
| 2 h | 0.142 | 0.108 | 0.137 | 0.116 | 0.152 | 0.149 |
| 4 h | 0.203 | 0.115 | 0.199 | 0.162 | 0.217 | 0.219 |
| 6 h | 0.245 | 0.147 | 0.248 | 0.218 | 0.256 | 0.252 |
| 8 h | 0.311 | 0.186 | 0.320 | 0.294 | 0.303 | 0.308 |
| 10 h | 0.342 | 0.222 | 0.330 | 0.309 | 0.341 | 0.328 |
| 1j 2 h | 0.365 | 0.324 | 0.342 | 0.338 | 0.348 | 0.351 |
| 1j 4 h | 0.354 | 0.332 | 0.351 | 0.356 | 0.336 | 0.342 |
| 1j 8 h | 0.337 | 0.334 | 0.342 | 0.333 | 0.315 | 0.324 |

TABLE 2
Measurement of OD₆₀₀ₙₘ of M. magneticum ABM-1 parent strain
(Control) or strains expressing pseudopalyne (paND) or staphylopine
(saEND) in the presence or absence of cobalt.

| | Control | Control | paND | paND | saEND | saEND |
|---|---|---|---|---|---|---|
| OD₆₀₀ₙₘ | 0 μM | 100 μM | 0 μM | 100 μM | 0 μM | 100 μM |
| 0 | 0.03 | 0.030 | 0.027 | 0.028 | 0.031 | 0.03 |
| 2 h | 0.031 | 0.027 | 0.032 | 0.030 | 0.033 | 0.032 |
| 4 h | 0.042 | 0.031 | 0.041 | 0.032 | 0.039 | 0.038 |
| 6 h | 0.047 | 0.036 | 0.049 | 0.039 | 0.048 | 0.046 |
| 8 h | 0.054 | 0.041 | 0.053 | 0.048 | 0.05 | 0.054 |
| 10 h | 0.063 | 0.046 | 0.066 | 0.057 | 0.061 | 0.060 |
| 1j 2 h | 0.096 | 0.082 | 0.102 | 0.094 | 0.092 | 0.088 |
| 1j 4 h | 0.098 | 0.089 | 0.099 | 0.097 | 0.095 | 0.096 |
| 1j 8 h | 0.095 | 0.093 | 0.097 | 0.101 | 0.092 | 0.094 |

TABLE 3
Measurement of OD₆₀₀ₙₘ of M. gryphiswaldense MRS-1 parent strain
(Control) or strains expressing pseudopalyne (paND) or staphylopine
(saEND) in the presence or absence of nickel.

| | control | control | paND | paND | saEND | saEND |
|---|---|---|---|---|---|---|
| OD₆₀₀ₙₘ | 0 mM | 1 mM | 0 mM | 1 mM | 0 mM | 1 mM |
| 0 | 0.104 | 0.106 | 0.119 | 0.118 | 0.117 | 0.117 |
| 2 h | 0.154 | 0.132 | 0.198 | 0.165 | 0.184 | 0.159 |
| 4 h | 0.268 | 0.156 | 0.334 | 0.240 | 0.278 | 0.216 |
| 6 h | 0.371 | 0.145 | 0.405 | 0.285 | 0.401 | 0.218 |
| 8 h | 0.458 | 0.153 | 0.502 | 0.296 | 0.511 | 0.226 |
| 10 h | 0.515 | 0.150 | 0.471 | 0.330 | 0.435 | 0.225 |
| 1j | 0.610 | 0.148 | 0.422 | 0.351 | 0.443 | 0.253 |
| 1j 2 h | 0.578 | 0.154 | 0.452 | 0.362 | 0.439 | 0.246 |
| 1j 4 h | 0.597 | 0.151 | 0.456 | 0.359 | 0.440 | 0.255 |

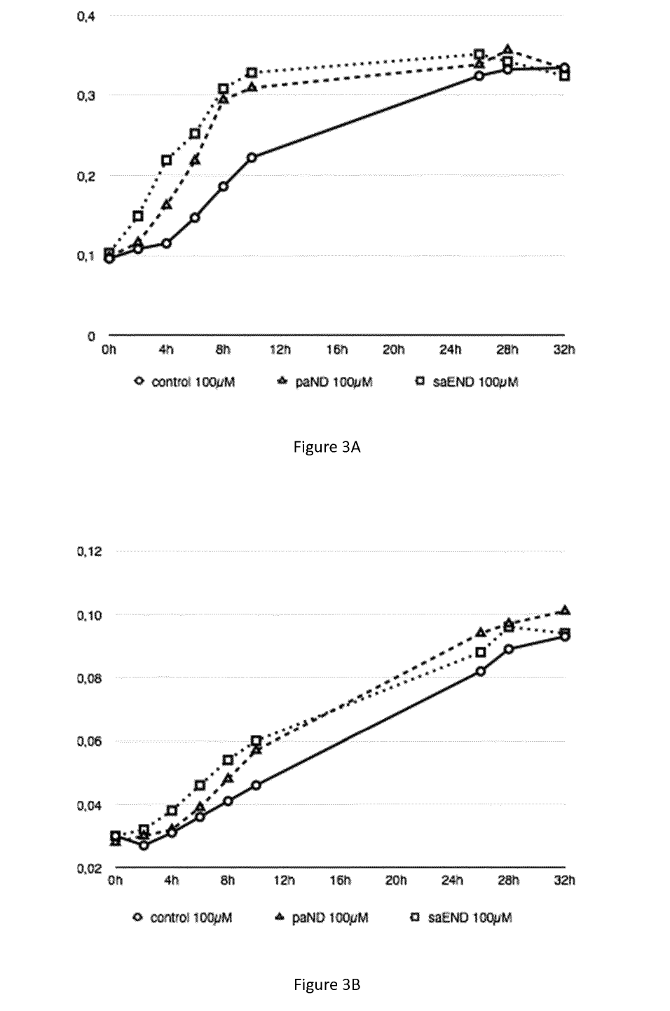

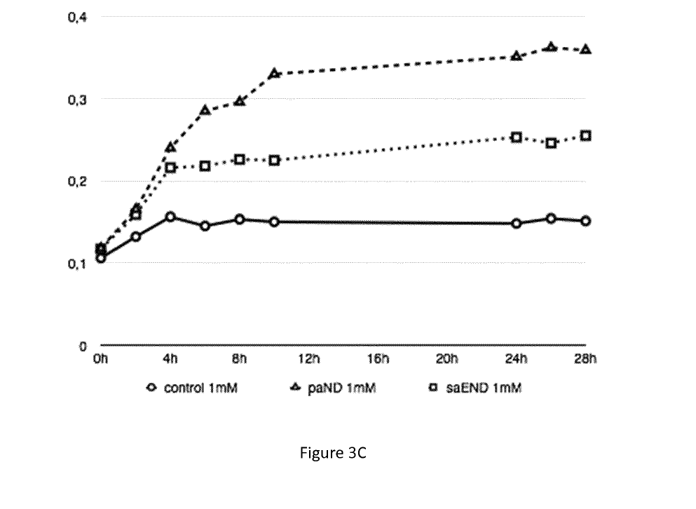

From FIG. 3A, FIG. 3B, and FIG. 3C and Tables 1, 2 and 3, it is concluded that magnetotactic bacteria have an increased resistance towards cobalt when producing pseudopaline or staphylopine. This difference of resistance is especially high between 6 h and 12 hours of culture in the presence of metal compared to culture conditions without metal.

In the case of resistance toward nickel, strain MSR-1 show a better growth when producing pseudopaline than when producing staphylopine.

Both strains, in the presence of cobalt or nickel, present better growth than the control strain.

The magnetotactic strains expressing the genes coding for the biosynthesis of pseudopaline and staphylopine resist to higher concentration of nickel and cobalt.

### Example 4

### Accumulation of Metal in Magnetotactic Bacteria

Magnetotactic strains have been cultivated in the appropriate medium (at least 200 mL) in the presence of cobalt (100 μM CoCl₂) or nickel (500 μM NiCl₂). Bacteria have then been collected by centrifugation and resuspended in a washing buffer (Tris 100 mM, glucose 10 mM). After centrifugation, the cell pellet has been dried at 70° C. overnight, weighted on a precision balance and dissolved in 5% nitric acid. Accumulated metal was measured by ICP-AES and the data are expressed as a function of the dry weight of the cell pellet.

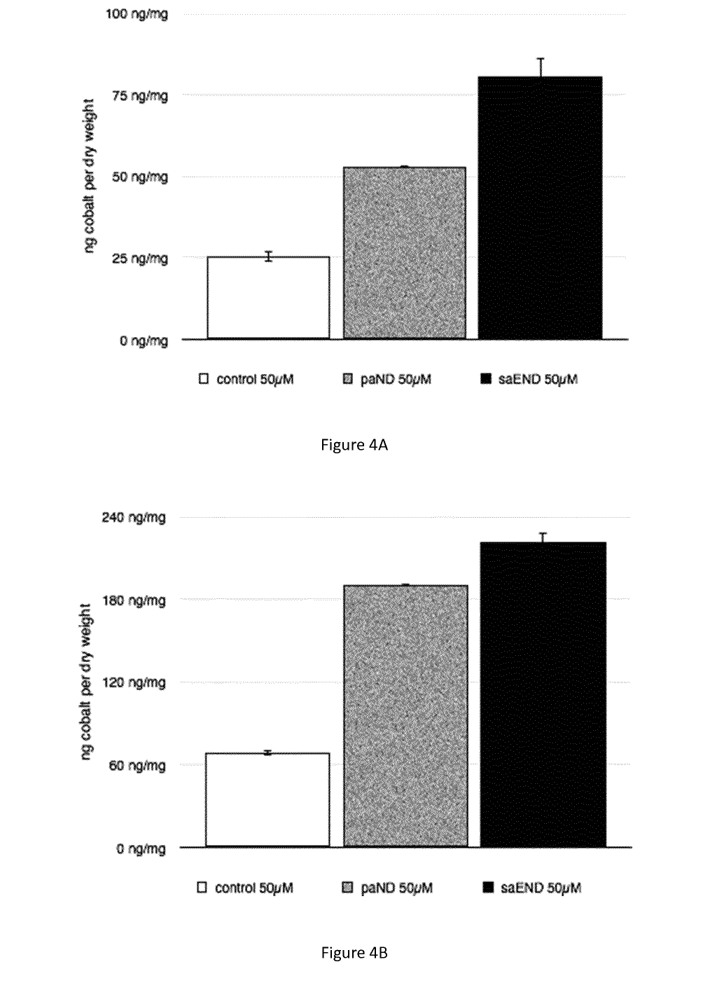

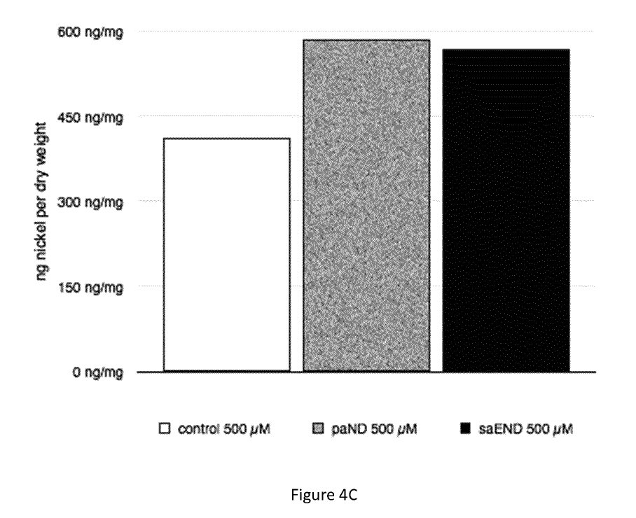

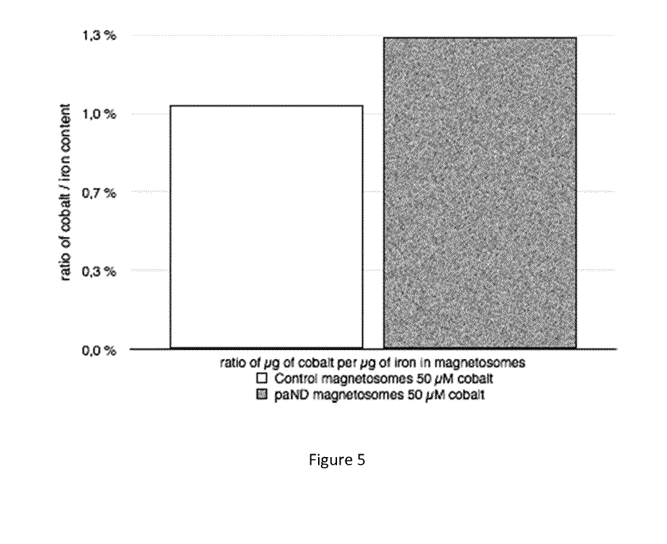

Data from FIG. 4A, FIG. 4B, and FIG. 4C show that magnetotactic bacterial strains (AMB-1 and MSR-1) expressing the genes responsible for the biosynthesis of pseudopaline and staphylopine accumulate more cobalt and nickel than the control strains that do not express these genes. More precisely, strain MSR-1 producing pseudopaline or staphylopine accumulates respectively two and three times more cobalt than the control strain (FIG. 4A). The same trend is observed in strain AMB-1 with an even higher accumulation for the strain producing pseudopaline (FIG. 4B). With regard to nickel the strain MSR-1 producing pseudopaline or staphylopine accumulates 150 to 160% more nickel than control strain (FIG. 4C).

### Example 5

### Cobalt Doping of Magnetosome

M. gryphyswaldense MSR-1 strains have been cultivated in 1.5 L of lactate medium in the presence of cobalt (100 μM CoCl₂). Bacteria have been then collected by centrifugation, and washed in the washing buffer. The cells have then been resuspended in 10 mL of resuspension buffer (HEPES 20 mM, NaCL 0.9% EDTA 1 mM glycerol 8%)+antiprotease and then disrupted by using a French press operating at 10.000 psi. 1 mL of the cell lysate has been kept for ICP-AES analysis of accumulated metals. The magnetosomes have been extracted from the rest of the lysate by simple magnetization, and washed 5 times with the resuspension buffer and then 5 times in using the same buffer except EDTA.

The final magnetosome resuspension has been eluted in 500 μL of an elution buffer (HEPES 20 mM glycerol 8%) and a fraction has been analyzed by ICP-AES. The content of cobalt in these magnetosomal preparations has been evaluated by comparison to the iron content.

TABLE 4
Cobalt and iron content of magnetosomes extracted

| | Cobalt | Iron | Cobalt per |
|---|---|---|---|
| | content | content | iron |
| | in μg | in μg | content |
| Magnetosome control; | 6.6 μg | 633.04 μg | 1.05% |
| 50 μM cobalt | | | |
| Magnetosome paND; | 9.03 μg | 710.72 μg | 1.28% |
| 50 μM cobalt | | | |

Magnetosomes extracted from those samples have a cobalt to iron ratio of 1.05% in the control conditions, and 1.28% when using the pseudopaline producing strain. This corresponds to a 20% increase in cobalt accumulated inside the magnetosomes when the bacteria produce pseudopaline.

### Example 6

### Repartition of Cobalt Between the Cytosolic and Magnetosomal Fractions

M. gryphyswaldense MSR-1 strains producing staphylopine have been cultivated in lactate medium in the presence of cobalt (100 μM CoCl₂). Bacteria have been then collected by centrifugation, and washed in the washing buffer. The cells have then been resuspended in 10 mL of resuspension buffer (HEPES 20 mM, NaCL 0.9% EDTA 1 mM glycerol 8%)+antiprotease and then disrupted by using a French press operating at 10.000 psi. The magnetosomal fraction has been separated from the cytosolic fraction by simple magnetization, and washed 5 times with the resuspension buffer and then 5 times in using the same buffer except EDTA.

XANES spectra were recorded on biological samples (magnetosomal and cytosolic fractions prepared as described above) and on several cobalt containing references (Co(II)-acetylacetonate; Co(II)-glutathione; Co₃O₄, Co(II)-cysteine, Co(II)-nicotianamine, cobalamin (Vitamin B12), Co(II)-acetate, Co(II)-nitrate, Co(II)-phosphate and commercial CoFe₂O₄ pellets).

Data were collected at the Co K absorption edge (7.709 keV), by scanning in the energy range 7.65-7.90 keV (XANES) or 7.65-8.35 keV (EXAFS) with a nitrogen-cooled double crystal monochromator. Spectra were recorded in fluorescence mode, using the crystal analyzer spectrometer of CRG-FAME (BM30B) at ESRF operated in 7/8 bunches mode (200 mA). XANES spectra recorded on biological samples were analyzed by Linear combination fitting calculated using spectra of reference compounds.

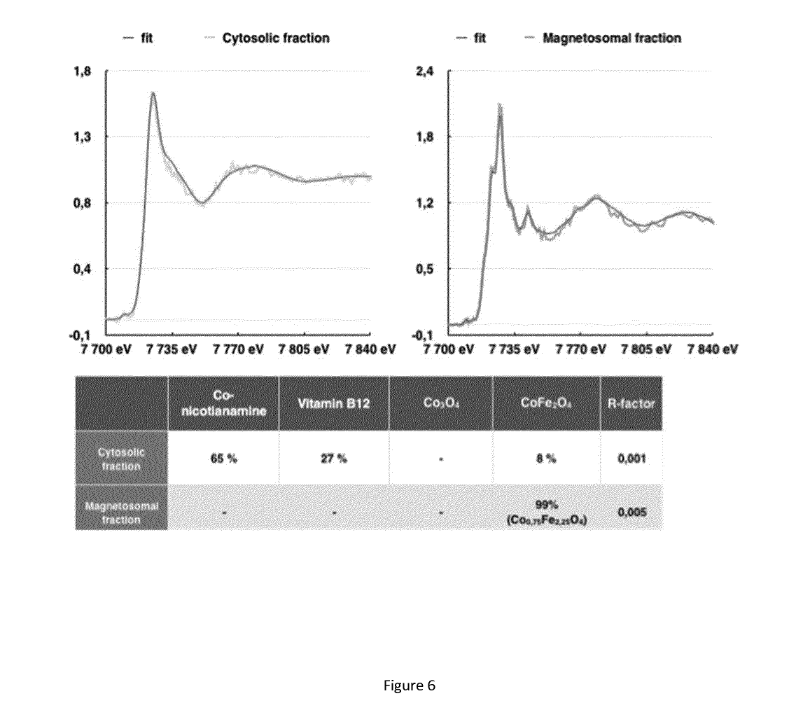

Results presented in FIG. 6 show that cobalt accumulates both in cytosol and in magnetosomes. In the cytosol, a fraction of the cobalt accumulated forms a complex with B12 vitamin (a cellular compound known to bind Co) but the majority of the metal is associated with the produced metallophore. In the magnetosome, the cobalt forms in majority a cobalt/iron complex, confirming an incorporation of cobalt into magnetite crystal.

### Example 7

### Characterization of Magnetotactic Bacteria Expressing Both a Metallophore and a Cobalt/Nickel-Specific Permease

a. Construction of the Plasmid for rpNxiA Expression

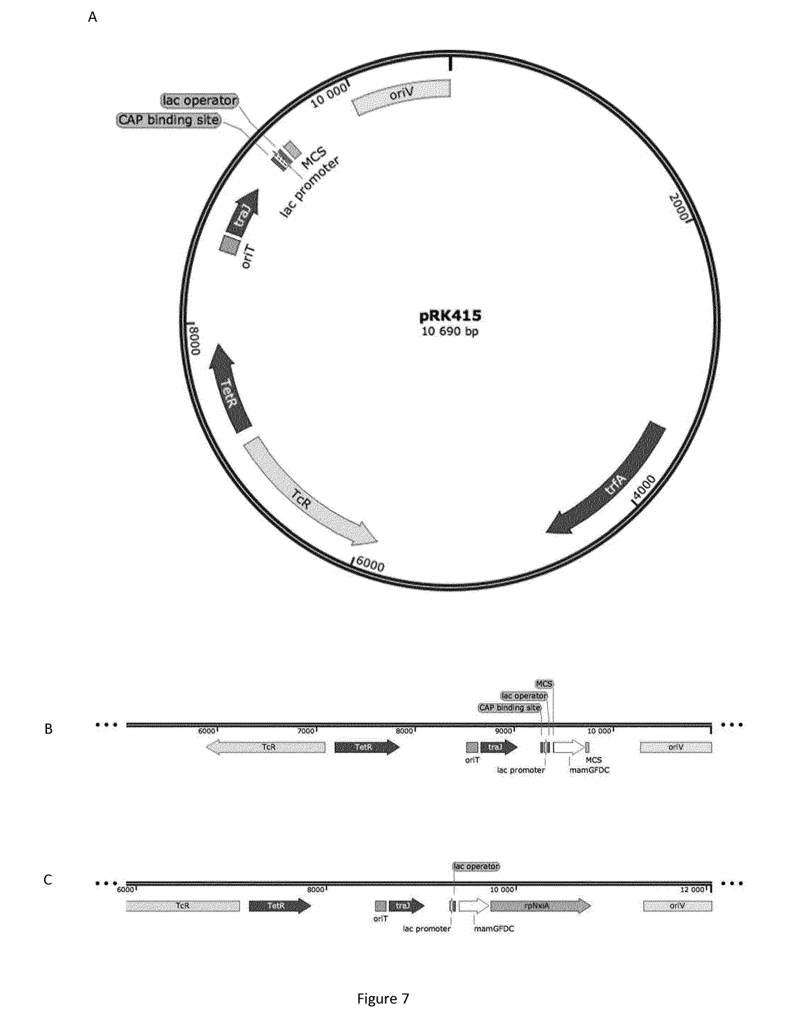

The vector used to express the permease was build using the pRK415 plasmid, as shown on FIG. 7, drawing A. The mamGFDC promotor was first amplified using genomic DNA from Magnetospirillum gryphiswaldense MSR-1 using Mam-F et Mam-R primers (Table 5). The rpNxia gene from Rhodopseudomonas palustris was amplified using the genomic DNA from R. palustris strain CGA009 with the primers RpNxia-F and RpNxia-R (Table 5). The sequence of the rpNxiA gene corresponds to SEQ ID NO:10

The mamGFDC promotor was subsequently cloned into prK415 using HindIII and BamHI (New England Biolabs ©) as restriction enzymes, resulting in the prK415-mam vector (FIG. 7, drawing B). Then, the rpNxiA gene was cloned in prK415-mam using KpnI and BamHI as restriction enzymes. The final plasmid is shown on FIG. 7, drawing C.

TABLE 5
Primers used to construct the final plasmid
expressing rpNxiA

| | | SEQ |
|---|---|---|
| | | ID |
| Primer | Sequence | NO: |
| Mam-F | CTCGAGGAGCTCAAGCTTTTCCAATGACCACCA | 11 |
| | CCAC | |
| Mam-R | GTCGACGGATCCACTAGTCTGATCTCCGGC | 12 |
| RpNxia- | ACTAGTGGATCCATGACCGATCTCGTTC | 13 |
| F | | |
| RpNxia- | GGTACCGAATTCTCATTTCTGCACGGCC | 14 |
| R | | |

b. Resistance to Metal

The phenotypes associated with the presence of the permease and/or a metallophore were determined using recombinant M. gryphiswaldense MSR-1 cells cultivated in appropriated medium in the absence or presence of metals (100 μM cobalt or 1 mM nickel). The growth curves were obtained by following the OD each 2 hours during 48 H.

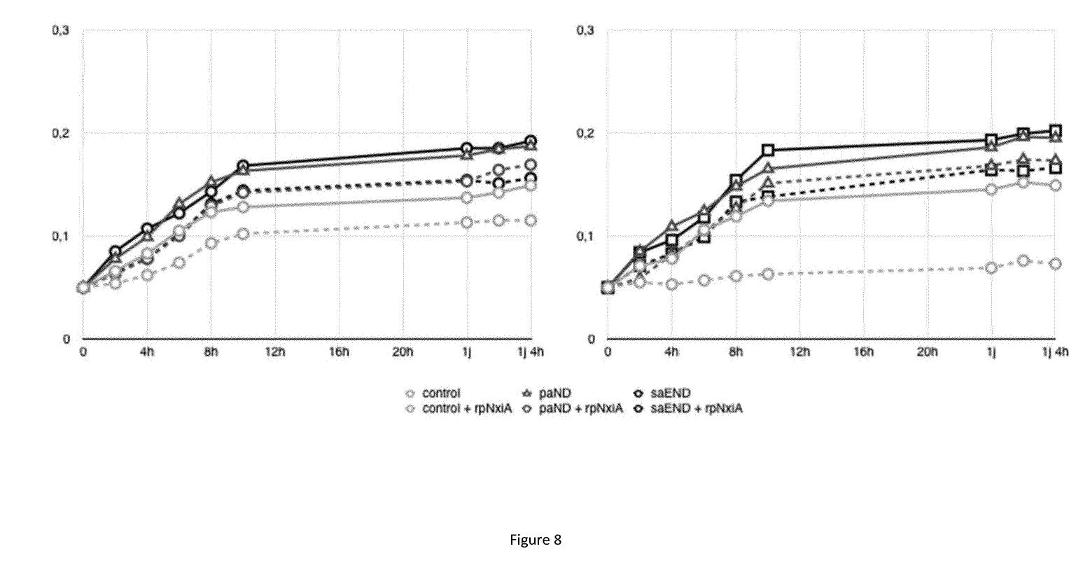

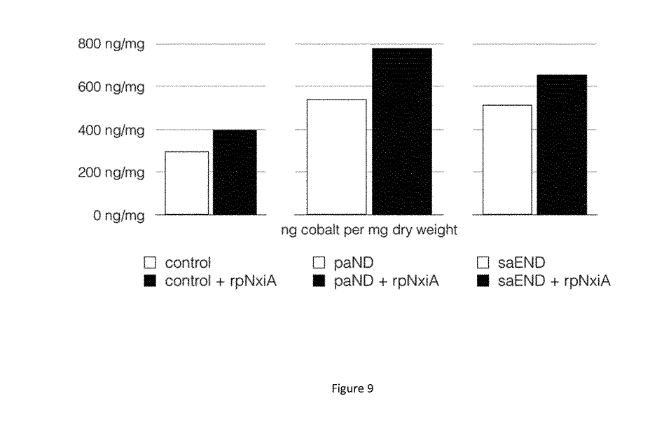

Data of FIG. 8 show that the expression of the permease alone increases the sensibility of the magnetotactic bacteria to metal toxicity, both to cobalt (A) and to nickel (B) compared to the control strain. Further, this figure shows that the strain expressing both a metallophore and a permease is less resistant to metal than the strain expressing a metallophore alone, but more resistant than the control strain. This effect is not aberrant since permeases are known to increase the accumulation of metal but at the same time, the sensibility to metal.

This result differs from the result obtained with metallophores which allow to increase both the accumulation of metal and the resistance.

c. Accumulation of Metal

In order to measure the quantity of cobalt accumulated, M. gryphiswaldense MSR-1 cells were incubated in the appropriate growth medium in the presence of 100 μM cobalt. After 24 H, cells were pelleted and washed (three times) using a washing buffer (Tris 100 mM glucose 10 mM pH 7.0). Cell pellets were then dried overnight and weighted before mineralization by addition of nitric acid (5%). Cobalt was subsequently quantified using ICP-AES.

FIG. 9 shows a higher accumulation of cobalt in strain expressing a permease versus a control strain (A). Further, it shows that accumulation is higher in strain expressing both a metallophore and a permease (B and C); this result is obtained both with staphylopine and pseudopaline. The accumulation of cobalt in the strain expressing both a metallophore and a permease is increased by at least a factor 2 (+50%).

1. A genetically modified magnetotactic bacterium that expresses a cobalt and/or nickel-specific metallophore.

2. The bacterium of claim 1, wherein the metallophore is selected from the group consisting of staphylopine and pseudopaline, and
a. when the metallophore is staphylopine, said bacteria expresses the proteins of SEQ ID NO:1, SEQ ID NO:2 and SEQ ID NO:3 or variants thereof, and
b. when the metallophore is pseudopaline, said bacteria expresses the proteins of SEQ ID NO: 4 and SEQ ID NO: 5 and variants thereof.

3. The bacterium of claim 1, wherein the cobalt and/or nickel accumulation in said bacterium is increased by at least 20% compared the cobalt and/or nickel content in the parent bacteria.

4. The bacterium of claim 1, wherein said bacterium contains at least 50 ng of cobalt by mg of dry weight.

5. The bacterium of claim 1, wherein said bacterium further expresses a cobalt and/or nickel permease.

6. The bacterium of claim 5, wherein said bacterium presents an increased capacity of resistance to cobalt and/or nickel and an increased capacity of accumulation of cobalt and/or nickel, both capacities being increased by at least 50% compared to the capacities of the parent bacterium.

7. The bacterium of claim 1, wherein said bacterium further comprises a reporter construct comprising a promoter sensitive to cobalt and/or nickel.

8. A cobalt and/or nickel-doped magnetosome having a cobalt/iron ratio of at least 1.25.

9-10. (canceled)

11. A method of bioremediating cobalt and/or nickel comprising contacting the cobalt and/or nickel with the bacterium of claim 1.

12. A method of detecting trace amounts of cobalt and/or nickel in a sample comprising contacting the sample with the bacterium of claim 1.

13. A process of recovering cobalt and/or nickel, comprising the steps of:
(i) contacting the bacteria of claim 1 with a medium containing cobalt and/or nickel,
(ii) incubating for a period of time, and
(iii) recovering bacteria containing cobalt and/or nickel with a magnetic field.

14. The process of claim 13, wherein said medium containing cobalt and/or nickel is a medium to be depolluted.

15. A method of treating a tumor in human subject in need of treatment comprising administering the magnetosome of claim 8 to the human subject.

16. A method of imaging cell or molecule of interest wherein the magnetosome of claim 8 is used as a contrast agent.
